(19) United States
(12) Patent Application Publication
(10) Pub. No.: US 2021/0147811 A1
(43) Pub. Date: May 20, 2021

(54) METHODS FOR IN VITRO EXPANSION OF ADULT TISSUE STEM CELLS

(71) Applicant: CEDARS-SINAI MEDICAL CENTER, Los Angeles, CA (US)

(72) Inventors: Barry STRIPP, Malibu, CA (US); Apoorva MULAY, Los Angeles, CA (US); Changfu YAO, Arcadia, CA (US)

(73) Assignee: Cedars-Sinai Medical Center, Los Angeles, CA (US)

(21) Appl. No.: 17/043,888

(22) PCT Filed: Apr. 9, 2019
(86) PCT No.: PCT/US2019/026654

§ 371 Date: Sep. 30, 2020

Related U.S. Application Data
(60) Provisional application No. 62/655,115, filed on Apr. 9, 2018.

(51) Int. Cl. C12N 5/071; A61K 35/42

(52) U.S. Cl. CPC C12N 5/0689; C12N 2501/999; A61K 35/42

(57) ABSTRACT

Described herein are methods and compositions for regulation of the p53 pathway, providing intrinsic “sternness” allowing for their efficient in vitro expansion following isolation. Pharmacologic approaches to modulate p53 signaling supports expansion of stem cells, including greater clonal expansion of lung stem cells when compared to use of other small molecules such as ROCK inhibitor Y27632 alone. Effects of combined treatment with Pifithrin-α and Y27632 are additive. The current invention involves use of drugs that target the p53 pathway to reversibly regulate stem cell expansion in vitro for banking of stem cells and for pre-conditioning of stem cells prior to orthotopic transplantation.

### STATEMENT REGARDING FEDERALLY-SPONSORED RESEARCH

This invention was made with government support under HL135163 awarded by the National Institutes of Health. The government has certain rights in the invention.

### FIELD OF THE INVENTION

The present invention relates to the field of culturing cells, and in particular, adult tissue stem cells in the lung epithelium.

### BACKGROUND

Cystic fibrosis is a monogenic disorder affecting approximately 1 in 2500 births or an estimated 70,000 individuals world-wide. The underlying genetic defect involves mutations that impact functionality of the Cystic Fibrosis Transmembrane Conductance Regulator (CFTR) leading to defects in electrolyte transport. The most pronounced clinical manifestations of cystic fibrosis result either from epithelial dysfunction and mucus plugging in many different tissues including the upper and lower respiratory tract, gastrointestinal tract, and reproductive tract, or from defects in nutrient and salt absorption/secretion. The advent of novel pharmacologic approaches to modulate CFTR trafficking or function has provided effective therapeutic options for approximately 75% of patients. However, improved options that go beyond palliative therapy are desperately needed for the thousands of patients that are not responsive to CFTR modulator therapy.

A significant fraction of patients who are not responsive to CFTR modulator therapies rely upon symptom management to slow declines in lung function. Cell- or gene-based therapies remain a viable option for the management of lung disease and long-term correction of the airway defect in cystic fibrosis (CF) patients. These approaches will require gene correction or replacement of region-specific airway stem/progenitor cells. Studies have shown that the epithelial lining of airways is maintained by region-specific stem cells whose positional identity is a critical determinant of differentiation potential and the types of specialized epithelial cells generated during homeostatic maintenance or repair. Recent reports suggest exogenously delivered lung-derived stem cells can both engraft and expand within suitably conditioned recipient tissue to replace depleted endogenous stem cells, and raise the compelling possibilities of cell-based therapies. However, a number of basic questions must be addressed to fully assess therapeutic potential of transplanted exogenously derived lung stem cells. Thus, there is a great need in the art for understanding the role of stem cells in the airway, and potential mechanisms for exploiting the regenerative properties of these cells.

Described herein are compositions and methods related to use of ROCK inhibitors, p53, and combinations thereof block apoptosis of lung stem cells, including freshly isolated cells, to enhance their “sternness”. p53 inhibitors are more effective than ROCK inhibitors at promoting the clonal expansion of lung stem cells but that the effects of these inhibitors are additive. These results support development of new therapeutic avenues, such as stem cell therapies to achieve long-term correction of CF lung disease.

### SUMMARY OF THE INVENTION

Described herein is a method of culturing cells, including providing a quantity of human stem cells, or stem cell derived cells culturing in the presence of at least one molecule including: a RHO-kinase (ROCK) inhibitor, SMAD inhibitor, and p53 inhibitor and expanding the quantity of stem cells, or stem cell derived cells. In other embodiments, the stem cells or stem cell derived cells are added to a media including at least one molecule including: a RHO-kinase (ROCK) inhibitor, SMAD inhibitor, and p53 inhibitor. In other embodiments, the RHO-kinase inhibitor is Y27632. In other embodiments, the p53 inhibitor is Pifithrin α. In other embodiments, the SMAD inhibitor is SB 431542. In other embodiments, the stem cells are adult stem cells. In other embodiments, the stem cell derived cells are lung epithelial cells. In other embodiments, the stem cell derived cells are organoids. In other embodiments, expanding the quantity of stem cells or stem cell derived cells includes increased proliferation of the cells. In other embodiments, expanding the quantity of stem cells or stem cell derived cells includes increased size of organoids including stem cell derived cells. In other embodiments, the expanding the quantity of stem cells or stem cell derived cells includes increased colony forming efficiency. In other embodiments, the at least one molecule includes Y27632 and Pifithrin α.

Further described herein is a cell culture including a quantity of human stem cells, or stem cell derived cells in a cell culture media including at least one molecule including: a RHO-kinase (ROCK) inhibitor, SMAD inhibitor, and p53 inhibitor. In other embodiments, the RHO-kinase inhibitor is Y27632 and the p53 inhibitor is Pifithrin α. In other embodiments, the stem cells are adult stem cells. In other embodiments, the stem cell derived cells are lung epithelial cells.

Also described herein is a method of treatment, including administering to a human subject afflicted with cystic fibrosis, a composition including a quantity of human stem cells or stem cell derived cells, wherein the quantity of human stem cells or stem cell derived cells have been cultured in the presence of at least one molecule including: a RHO-kinase (ROCK) inhibitor, SMAD inhibitor, and p53 inhibitor prior to administering to the human subject. In other embodiments, the RHO-kinase inhibitor is Y27632 and the p53 inhibitor is Pifithrin α. In other embodiments, the stem cells are adult stem cells. In other embodiments, the stem cell derived cells are lung epithelial cells.

### BRIEF DESCRIPTION OF FIGURES

FIG. 1. Experimental design. Preconditioning of donor cells, including addition of small molecules such as ROCK inhibitor, pifithrin α, or combined together, and generation of organoids.

FIG. 2. Freshly isolated cells cultured with small molecules at different time points. As shown, both ROCK inhibitor and pifithrin α, have an effect on proliferation of cells, with a synergistic effect being observed for their combined use.

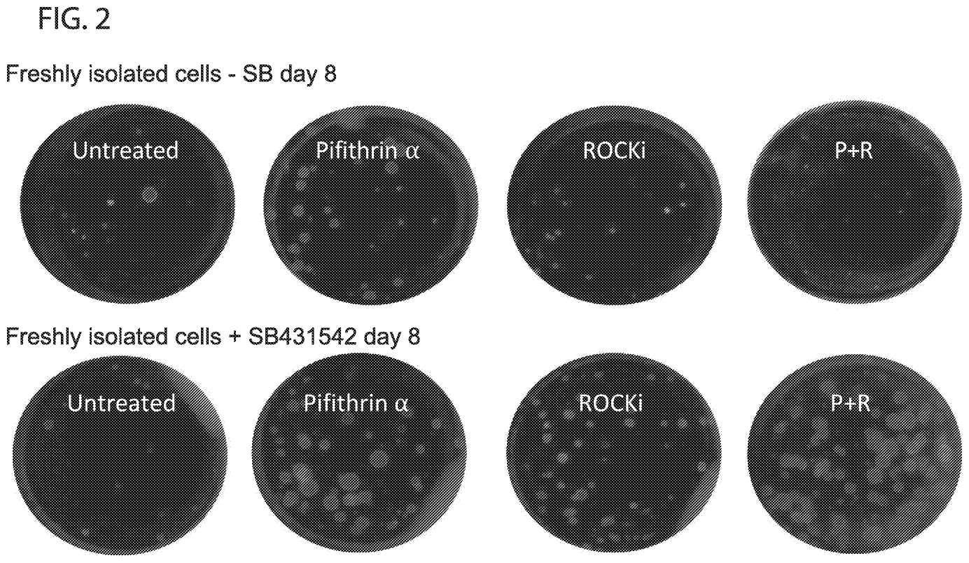

FIG. 3. Synergistic effect of small molecules. Addition of the TGF-β inhibitor, SB 431542 to the culture medium increases the % colony forming efficiency of freshly isolated murine lung epithelial cells. Addition of Pifithrin α causes an increase in organoids size, while addition of ROCKi causes an increase in the colony forming efficiency.

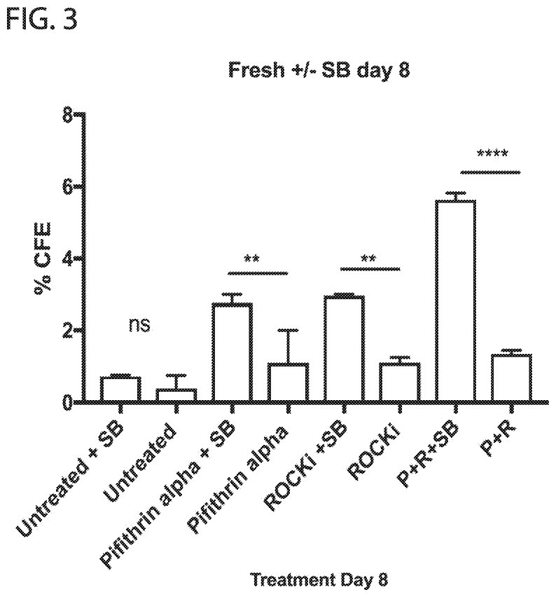

FIG. 4. Effect of pre-culturing—Day 3. Pre-culturing cells on collagen coated plates for 24 hrs after initial isolation increases the rate of organoid formation, with organoids being visible as early as day 3 in culture.

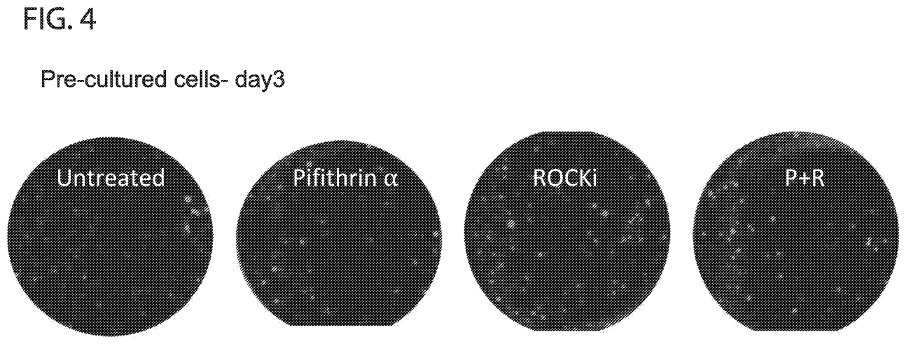

FIG. 5. Effect of pre-culturing—Day 8. Addition of Pifithrin α causes an increase in organoids size, while addition of ROCKi causes an increase in the colony forming efficiency. This effect is more pronounced when cells are pre-cultured for 24 hrs on collagen coated plates after initial isolation.

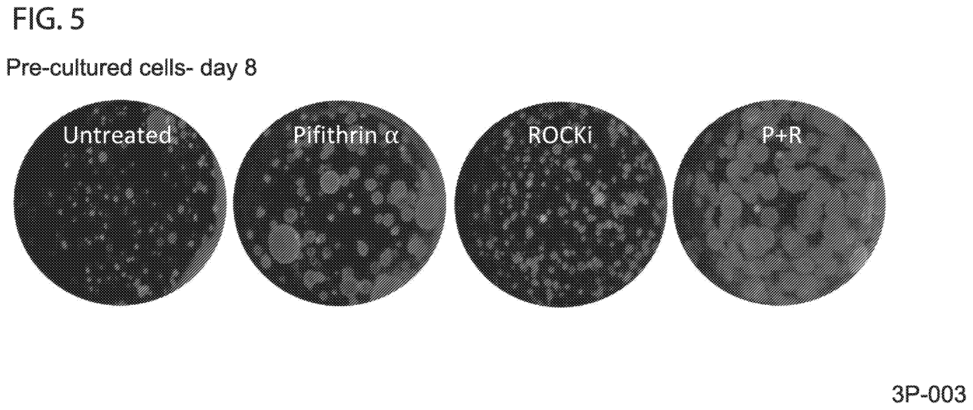

FIG. 6. Effect of small molecules on cultured cells—Day 3 and 8.

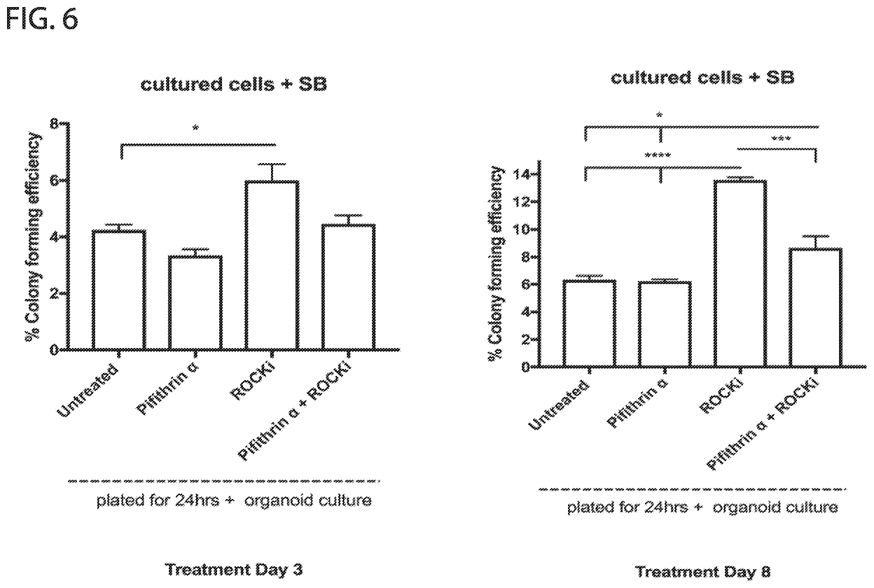

FIG. 7. Comparison of fresh and cultured cells—Day 8.

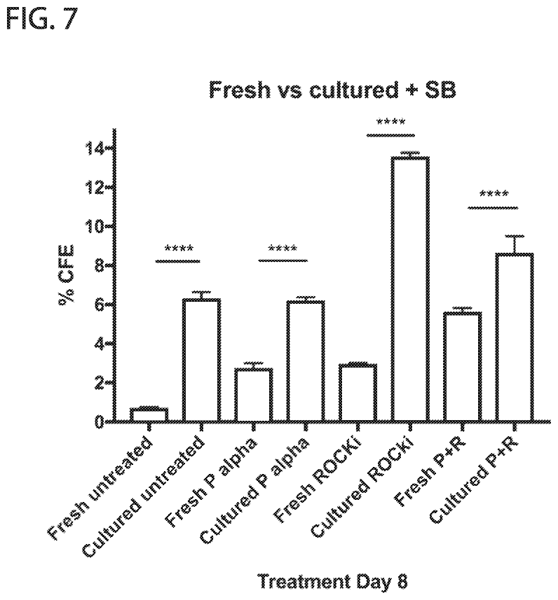

FIG. 8. Exposure to total body X-Rays leads to a dose-dependent decrease in epithelial colony-forming efficiency (CFE). ROSA-RFP were exposed to increasing doses of total body X-Rays 24 hours prior to cell isolation. Epithelial cells were isolated from lungs of exposed ROSA-RFP mice and unexposed control ROSA-GFP mice, mixed in equal proportions and plated in 3D MatriGel cultures with 1×10⁵ stromal support cells. Organoids were cultured for 14 days. Left panels show fluorescence imaging of representative wells with quantitative data shown in the graph on the right. Whole body X-Ray exposure led to a dose-dependent decrease in epithelial CFE, which decreased by >95% at 6 Gy and above.

FIG. 9. Orthotopic cell transplantation. Scgb1a1-HSVtk transgenic mice were exposed to gancyclovir delivered through miniosmotic pump and club cell ablation monitored by immunofluorescence. A dose of 12.5 mg gancyclovir (total delivered dose) led to >90% loss of Scgb1a1 immunoreactive cells 7 days post-treatment (red immunofluorescence in panel A). Total lung cells were isolated from ROSA-GFP mice and 1×10⁶ cells transplanted into lungs of gancyclovir-treated mice that received i.t. chlodronate-containing liposomes for depletion of macrophages. Transplanted cells are shown in green in B and C, with cell type-specific markers shown by red immunofluorescence in C. Transplantation of 1×10⁶ ROSA-mT lung cells (total cells following depletion of CD45+ fraction) into recipient lung tissue pre-conditioned by 6 Gy X-Rays led to efficient engraftment as shown by light sheet microscopy 2 weeks post transplant (D).

FIG. 10. p53 loss-of-function in airway progenitor cells SB431542 increases colony-forming efficiency (CFE).

FIG. 11. Rho kinase inhibitor HA1077 increases in vitro clonogenic potential without altering colony-forming ability.

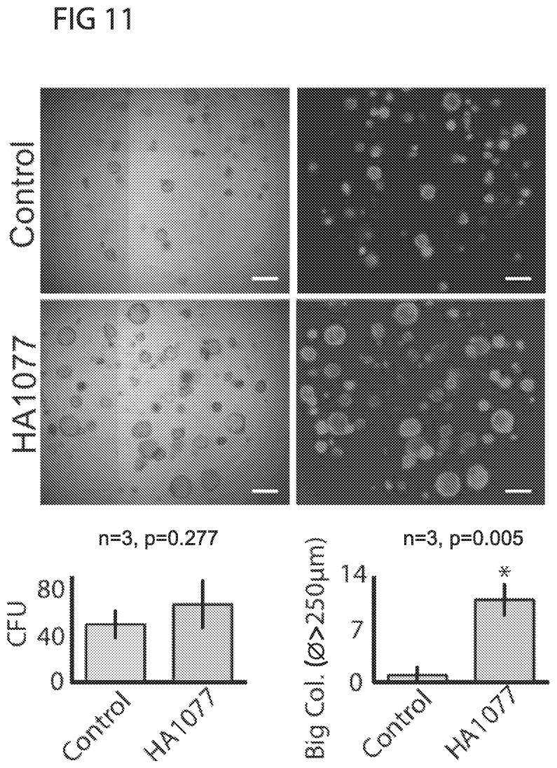

FIG. 12. Background. The cellular composition of the epithelium changes along the proximal-distal axis of the airways. Region specific stem/progenitor cell populations contribute to the maintenance and regeneration of different parts of the airways. In the mouse basal cells are mostly restricted to the trachea whereas in humans there is a diminishing gradient of basal cells along the proximal-distal axis

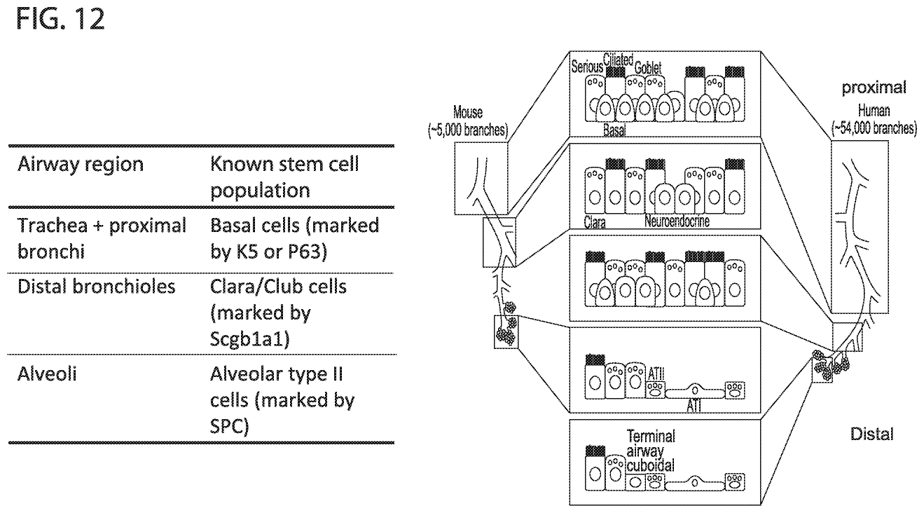

FIG. 13. Effect of p53 and ROCKi inhibition on murine freshly isolated distal total epithelial cells in (+SB431542). Addition of both ROCKi and Pifithrin alpha (in presence of SB431542) increases the colony forming efficiency of freshly isolated murine distal total epithelial cells (isolated from the lung without trachea). The effect is synergistic for their combined use, suggesting that they act through distinct mechanisms (A,B). Pifithrin alpha also causes an increase in the size of the organoids (C). Thus ROCKi seems to increase survival while Pifithrin alpha seems to increase survival and proliferation of the stem cells. The drop in mean diameter of the Pifithrin alpha+ROCKi group is likely due to space constraints because of the large number of organoids per well.

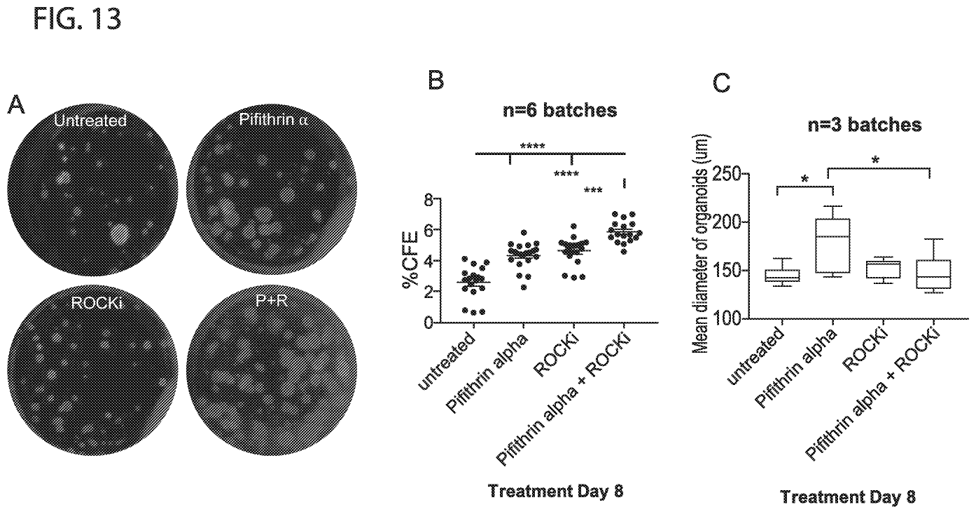

FIG. 14. Effect of inhibitors on murine pre-cultured distal total epithelial cells in (+SB431542). Addition Pifithrin alpha increases organoid size while addition of ROCKi increases the colony forming efficiency when murine distal total epithelial cells (isolated from the lung without trachea) are pre-cultured on collagen coated plates for 24 hrs prior after initial isolation. The effect on size is more pronounced when cells are precultured for 24 hrs on collagen coated plates after initial isolation. The drop in CFE in the Pifithrin alpha+ROCKi group is likely due space and nutrient availability constraints due to the large number of organoids per well.

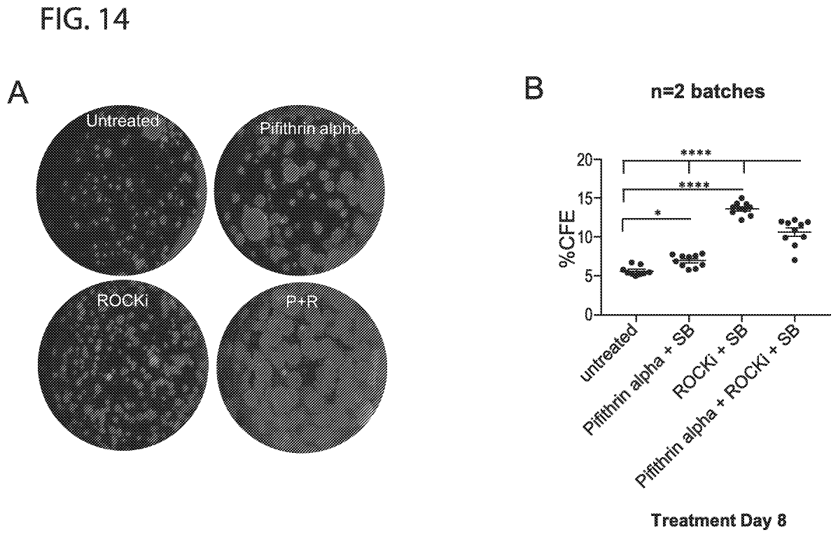

FIG. 15. Effect of ROCK and P53 inhibition on lineage labelled murine alveolar type II cells. GFP⁺ alveolar type II cells were isolated from tamoxifen treated SPC-CreER/ROSARG mice. Addition of both ROCKi and Pifithrin alpha (in presence of SB431542) increases the colony forming efficiency of freshly isolated alveolar typeII cells (regional stem cells which maintain the epithelium of the air sacs in the lung). The effect is synergistic for their combined use, similar to that seen for total distal epithelial cells in the previous figures. Addition of both ROCKi and Pifithrin alpha does not increase the size of the alveolar organoids (C)

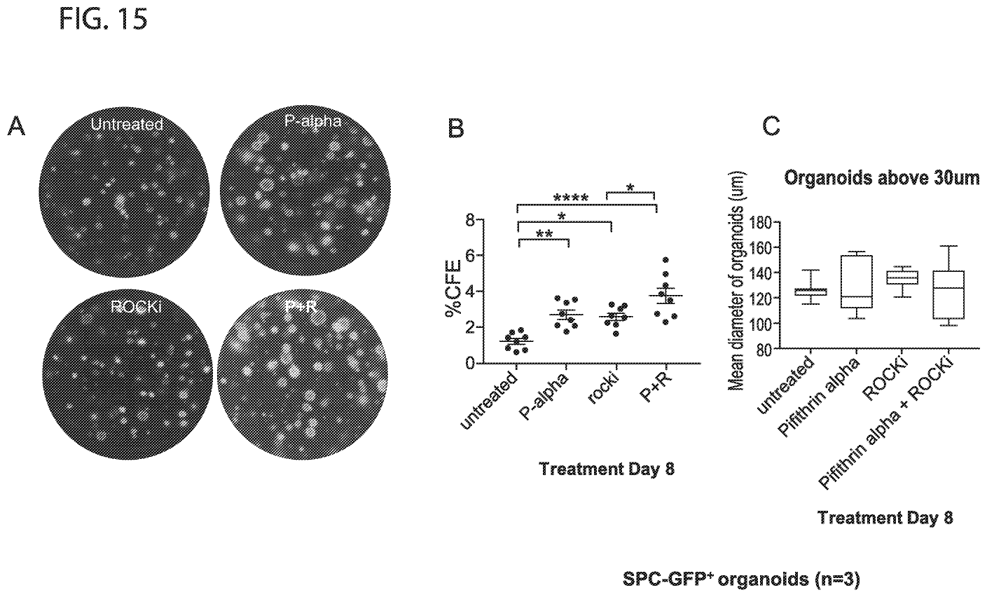

FIG. 16. Pifithrin alpha and ROCKi do not alter differentiation potential of distal progenitors. Distal progenitor cells were cultured as 3D organoids for 14 days in presence of Pifithrin-alpha, ROCKi and SB431542 followed by culture for 7 days in media without all three inhibitors. Addition of inhibitors did not alter the differentiation potential of the cells, as indicated by presence of Podoplanin (PDPN) positive alveolar type I cells (red) formed by differentiation of Surfactant Protein C (SPC) positive alveolar type II cells (green). DAPI (blue) is a nuclear stain.

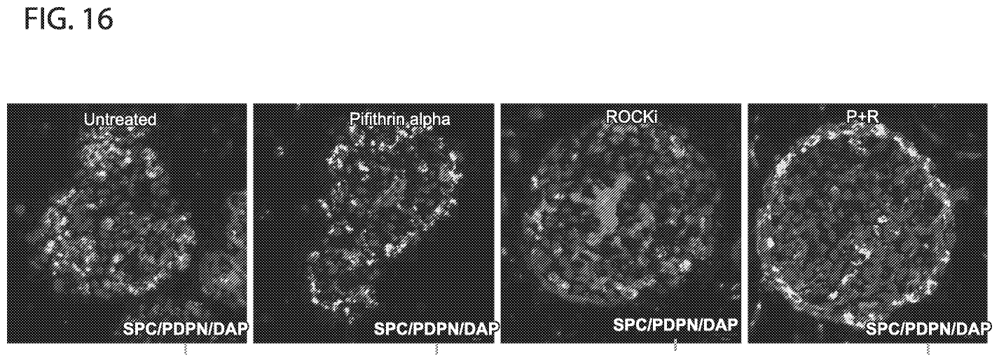

FIG. 17. Effect of ROCK and P53 inhibition on lineage labelled murine club cells. GFP⁺ club cells were isolated from tamoxifen treated Scgb1a1-CreER/ROSARG mice. Addition of ROCKi (in presence of SB431542) increases the colony forming efficiency of freshly isolated club cells (regional stem cells which maintain certain cell types in the bronchiolar epithelium) whereas addition of Pifithrin alpha (in presence of SB431542) increases the size of Scgb1a1-derived organoids.

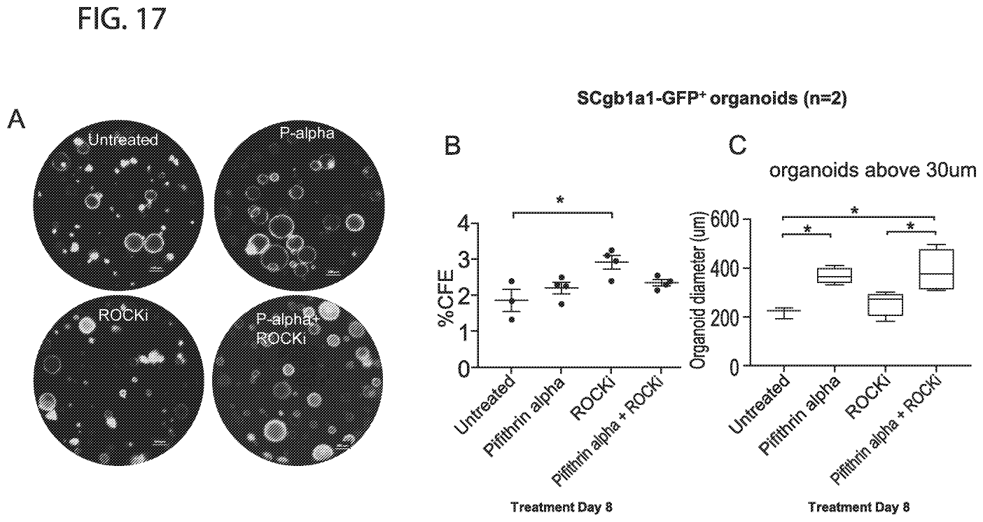

FIG. 18. Effect of ROCK and P53 inhibition on murine tracheal progenitor (basal) cells. Addition of both ROCKi (in presence of SB431542) increases the colony forming efficiency of freshly isolated murine proximal total epithelial cells (isolated from the murine trachea). However, addition of pifithrin alpha does not show a significant effect. Thus ROCKi seems to increase survival of both proximal and distal progenitors while the effect of Pifithrin alpha is specific to distal stem cells.

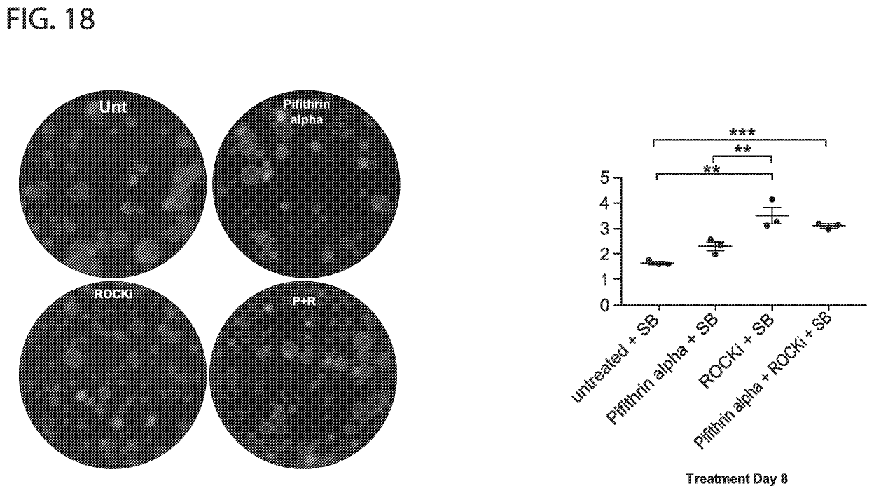

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed, Revised, J. Wiley & Sons (New York, N.Y. 2006); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

Without being bound by any particular theory, it is suggested that multiple distinct regional epithelial stem cells have potential to engraft within suitably pre-conditioned recipient lung tissue, that dependency on co-engrafting support cells can be overcome by pharmacologic blockade of anoikis and promotion of “sternness”, and that engrafting stem cells will generate specialized progeny that reflect their originating positional identity regardless of the site of engraftment within recipient tissue. These interrelated mechanisms can be evaluated by the following approaches.

First, conducting lineage tracing to define cellular interactions that promote epithelial stem cell engraftment. Co-engrafting structural lung cells may be necessary to enhance survival of engrafting epithelial stem cells and that these interactions can be restored following cell fractionation through recombination of fractionated lung cells. Lineage tracing in mice will be used to indelibly tag lung epithelium, stroma or endothelial cells. Either mixed cell populations or fractionated subsets will be transplanted into pre-conditioned lung tissue to determine cellular interactions that promote cell engraftment and stem cell expansion.

Second, conducting pharmacologic manipulation to enhance functional integration of transplanted epithelial progenitors. Importantly, it is suggested that co-engrafting structural lung cells can be dispensed with through pharmacologic inhibition of Rho kinase (ROCK) and/or p53 signaling, to block anoikis (programmed cell death due to extracellular matrix detachment) and enhance epithelial “sternness”. The impact of either ROCK and/or p53 inhibition prior to and during epithelial progenitor cell isolation will be determined by measuring in vitro organoid formation in addition to in vivo measures of engraftment and fate following transplantation.

Third, conducting lineage tracing and single cell RNA-Seq to define engraftment-competent epithelial stem cells and their fate. The Inventors will evaluate multiple epithelial stem cells residing in conducting airway and parenchymal regions can engraft within recipient lung tissue that has been pre-conditioned to functionally deplete resident stem cells. The Inventors will use lineage tracing in mice to fate map airway epithelium, airway club cells, airway basal cells or alveolar type 2 cells. The fate of lineage labeled epithelial cell populations will be traced either in their native state in uninjured mice or following isolation and transplantation into recipient mice whose lungs have been pre-conditioned to “accept” engrafting stem cells.

These approaches should serve to define epithelial stem cells of the mouse lung that can functionally engraft following transplantation, define cell types that interact to promote stem cell engraftment and validate pharmacologic strategies aimed at enhancing the engraftment potential of isolated lung stem cells. These studies will provide a knowledge base upon which to build stem cell therapies to achieve long-term correction of cystic fibrosis (CF) lung disease.

Stem cells are retained in vivo through interactions with their niche. These interactions are lost following isolation leading to their differentiation or apoptosis. Regulation of the p53 pathway provides intrinsic “sternness” allowing for their efficient in vitro expansion following isolation. Use of pharmacologic rather than genetic approaches to modulate p53 signaling allows for these effects to be reversed.

Methods have been developed for expansion of stem cells using a combination of irradiated NIH-3T3 fibroblasts and a ROCK inhibitor (Y27632). However, these methods are inferior to those developed using p53 inhibitors and result in the expansion of cells that show reduced capacity for differentiation. Preliminary studies by the Inventors have shown that pharmacologic inhibition of p53 results in greater clonal expansion of lung stem cells compared to use of ROCK inhibitor Y27632 alone. Effects of combined treatment with Pifithrin-α and Y27632 are additive. Future studies will determine reversibility of Pifirthrin-α effects on stem cell expansion and differentiation. This includes: 1) validating that the observed increase in clonogenic potential of lung stem cells is the result of stem cell expansion, 2) application of the aforementioned pharmacologic intervention during in vitro stem cell expansion enhances their viability following cryopreservation, 3) that application of the aforementioned pharmacologic intervention during in vitro stem cell expansion enhances to functionally engraft into lung tissue following orthotopic lung transplantation, 4) that stem cells expanded under these conditions retain full capacity for differentiation into specialized lung epithelial cell types and 5) that established culture conditions can be used to expand human lung stem cells. Various p53 inhibitors and/or downstream targets of p53 may also identify the potential for other activities of Pifithrin-α (independent of p53) in the regulation of stem cell behavior. To the Inventors' knowledge inhibitors of p53 have not been used for expansion of adult tissue stem cells. The Inventors' work was the first to show that the p53 pathway regulates stem cell quiescence in slowly regenerating tissues such as the lung. See McConnell et al., Cell Rep. 17:2173-2182, 2016, which is fully incorporated by reference herein. The current invention involves use of drugs that target the p53 pathway to reversibly regulate stem cell expansion in vitro for banking of stem cells and for pre-conditioning of stem cells prior to orthotopic transplantation

Stem cells maintain the epithelial lining of the lung, with important implications for cell-based therapy. Epithelial stem and progenitor cells contribute to lung morphogenesis during development, maintenance of the postnatal lung and repair following injury. The consensus from these studies is that multipotent endodermal progenitors of the developing lung give rise to lineage committed region-specific stem cells that maintain the epithelial lining of the postnatal lung. “Sternness” has been inferred in mouse models through use of lineage tracing to reveal cells capable of long-term self-renewal. Based upon this criterion, basal cells of pseudostratified airways, club cells of bronchioles and alveolar type 2 (AT2) cells of alveolar regions, serve as local stem cell populations.

However, even progenitor cells with limited lifespan under homeostatic conditions can be recruited to the stem cell pool in response to severe injury. This has been shown following genetic ablation of basal cells, wherein secretory cells can “dedifferentiate” to replace all cell types of the pseudostratified airway, and following severe virus-induced lung injury, wherein a distal airway Sox2-expressing progenitor yields ectopic basal cell-like progeny to replace injured airway and alveolar epithelium. Emerging concepts based upon these data are that many progenitor cell types, some of which do not fulfill the classical definition of a stem cell during homeostatic tissue maintenance, function as stem cells to repair severe tissue damage. Transplantation of either classical or facultative stem cells can have equal potential to repopulate the stem/progenitor cell depleted epithelium of recipient lung tissue.

Orthotopic cell transplantation to repair/replace epithelium of the diseased lung. The potential for therapeutic replacement of epithelial stem cells has gained increasing support for the treatment of intractable lung diseases for which few other options exist beyond lung transplantation. Clinical indications for such therapies might include replacement of defective epithelial cells with normally functioning counterparts, such as in patients with CF lung disease that is not responsive to currently available channel modulators. Three reports in the recent literature describe studies in mice aimed at generating stable orthotopic cell transplants that functionally replace injured lung epithelium. Common to each of these reports is the need for pre-conditioning of the recipient lung to create an injured tissue environment that is permissive for the engraftment of transplanted cells. Pre-conditioning regimens include naphthalene-induced lung injury (275 mg/kg), naphthalene (200 mg/kg) plus ionizing radiation (6 Gy total body gamma irradiaiton) and infection with mouse-adapted H1N1 influenza virus (PR8 strain). In addition to differences in preconditioning regimen, the identity, purity, route and fate of transplanted cells differ widely between studies with no consensus between the studies.

P53 and ROCK regulate epithelial colony-forming ability and “stemness”. Tp53 (p53) is a tumor suppressor that is one of the most commonly mutated genes in cancer. In addition to its classical functions of regulating cell fate following cellular stress, p53 regulates migration, autophagy, metabolism, and tumor microenvironment signaling. p53 regulates self-renewal and terminal differentiation of both neural and mammary stem cells in vitro, and of hematopoietic and kidney stem/progenitor cells in vivo. In the Inventors' previous work the Inventors have shown that loss of p53 function promotes stem cell renewal in vivo and leads to a dramatic increase in colony-forming epithelial progenitor cells in vitro. The impact of p53 loss-of-function on airway epithelial in vitro colony-forming ability can be phenocopied by pharmacologic inhibition of Rho kinase (ROCK). Experiments proposed in the Inventors' application will use transient pharmacologic modulation of ROCK and p53 as tools to enhance survival and “sternness” of transplanted epithelial cells and overcome the need for co-transplanted structural lung cells for their efficient engraftment.

Characterization of stem cell niches and therapeutic stem cell engraftment. As described, microenvironmental control appears important for supporting therapeutic stem cell engraftment: 1) Specifically, of interest is identifying the pre-conditioning regimen that impacts the identity of engrafting epithelial stem cells, the efficiency of engraftment and their subsequent fate. The Inventors will use state-of-the-art approaches to quantify and localize donor cell engraftment and determine their fate. These data provides insights into strategies that might be effectively used to promote efficient therapeutic stem cell engraftment to rectify electrolyte transport defects in the CF lung. 2) Extending those observations is understanding the contribution of intrinsic versus microenvironmental factors that dictate stem cell fate following engraftment. Through lineage tracing of donor cells the Inventors will be able to assess how intrinsic positional identity within donor lung tissue impacts stem cell fate following engraftment within “matched” versus “non-matched” microenvironments (i.e. relative to originating microenvironment of donor lung) within recipient lung tissue. 3) These results support definition of those cellular interactions within mixed populations of dissociated lung tissue that promote epithelial stem cell engraftment following transplantation. Furthermore, one can determine whether transient inhibition of either ROCK or p53 signaling can overcome the requirement for co-transplanted cells to promote efficient epithelial stem cell engraftment.

Development of long-term culture and tissue progenitor and iPSC differentiation protocols to reconstitute airway epithelium in vivo. Even though in vitro culture expansion of epithelial stem cells prior to transplantation has the potential to expand the pool of transplantable cells, the potential for culture bias in stem cell expansion and introduction of genomic instability leading to altered function has potential to bias interpretation.

Manipulation of stem cell microenvironment will serve to define engraftment-competent epithelial cells that can be recovered from adult donor lung tissue and strategies to enhance their functional integration within host tissue following transplantation by exploiting those finding related to modulating cell survival, engraftment and clonal expansion in orthotopic cell transplantation models. As p53 activity is believed to inversely correlate with “sternness” and in vitro colony-forming ability of lung stem cells. Transient inhibition of p53 signaling will be used to overcome stress-induced p53 activation associated with donor tissue dissociation that impacts in vitro organoid formation and may similarly affect survival, engraftment and clonal expansion following transplantation. This includes transient inhibition of ROCK, which has been shown to enhance survival, organoid formation and “sternness” of epithelial cells.

Replacement of defective cystic fibrosis airway epithelium with long-term repopulating epithelial stem cells represents a rational approach aimed at providing therapeutic options for patients who are not responsive to current CFTR modulator therapies. Early lung endoderm is multipotent but undergoes progressive lineage restriction to yield region-specific epithelial stem/progenitor cells that maintain the postnatal lung epithelium. Stem cell behavior is controlled through microenvironmental cues that include anatomic location within the airway. Progenitor cells can acquire “Sternness” following severe injury in vivo, by altering microenvironmental cues in vitro. The Inventors will test the notion that multiple epithelial progenitor cell types assume properties of stem cells following transplantation as a result of their engraftment within a permissive microenvironment within recipient tissue.

Described herein is a method of culturing cells, including providing a quantity of human stem cells, or stem cell derived cells culturing in the presence of at least one molecule including: a RHO-kinase (ROCK) inhibitor, SMAD inhibitor, and p53 inhibitor and expanding the quantity of stem cells, or stem cell derived cells. In other embodiments, the stem cells or stem cell derived cells are added to a media including at least one molecule including: a RHO-kinase (ROCK) inhibitor, SMAD inhibitor, and p53 inhibitor. In other embodiments, the RHO-kinase inhibitor is Y27632, HA1077, or other RHO-Kinase inhibitor known in the art. In other embodiments, the RHO-kinase inhibitor is Y27632. In other embodiments, the p53 inhibitor is Pifithrin α or other p53 inhibitors known in the art. In other embodiments, the p53 inhibitor is Pifithrin α. In other embodiments, the SMAD inhibitor is SB 431542 or other SMAD inhibitors known in the art. In other embodiments, the SMAD inhibitor is SB 431542. In other embodiments, the one of more molecules are added at 1, 2, 3, 4, 5, 6, 7 days, 1, 2, 3 weeks or more after fresh isolation of the cells from a human subject. In other embodiments, the one of more molecules are added for 1, 2, 3, 4, 5, 6, 7 days, 1, 2, 3 weeks. In various embodiments, the cells are precultured for about 24 hrs on collagen coated plates after initial isolation. In various embodiments, the quantity of human stem cells, or stem cell derived cells are culturing a cell culture media including at least one molecule including: a RHO-kinase (ROCK) inhibitor, SMAD inhibitor, and p53 inhibitor. In other embodiments, the RHO-kinase inhibitor is Y27632 and the p53 inhibitor is Pifithrin α.

In other embodiments, the stem cells are adult stem cells. In other embodiments, the stem cell derived cells are progenitor cells. In various embodiments, the stem cells are from organs where the epithelium is quiescent under homeostatic conditions and injury or infection can lead to the quiescent stem cell population to undergo expansion. In various embodiments, the stem cells are from lung, liver, kidney and intestine. In other embodiments, the stem cell derived cells are lung epithelial cells. In other embodiments, the stem cell derived cells are organoids. In other embodiments, expanding the quantity of stem cells or stem cell derived cells includes increased proliferation of the cells. In other embodiments, expanding the quantity of stem cells or stem cell derived cells includes increased size of organoids including stem cell derived cells. In other embodiments, the expanding the quantity of stem cells or stem cell derived cells includes increased colony forming efficiency. In various embodiments, the expanding the quantity of stem cells or stem cell derived cells includes an increase in proximal and/or distal cells. In various embodiments, the distal cells are alveolar type II progenitors and/or Scgb1a1 positive club progenitor cells. In other embodiments, the at least one molecule includes Y27632 and Pifithrin α. Also described herein is a quantity of organoids including cells made by the aforementioned methods. In various embodiments, the organoids include proximal and/or distal cells. In various embodiments, the distal cells are alveolar type II progenitors and/or Scgb1a1 positive club progenitor cells.

Further described herein is a cell culture including a quantity of human stem cells, or stem cell derived cells in a cell culture media including at least one molecule including: a RHO-kinase (ROCK) inhibitor, SMAD inhibitor, and p53 inhibitor. In other embodiments, the RHO-kinase inhibitor is Y27632 and the p53 inhibitor is Pifithrin α. In other embodiments, the stem cells are adult stem cells. In other embodiments, the stem cell derived cells are lung epithelial cells. In various embodiments, the aforementioned inhibitors are at a concentration of 0.1-1 uM, 1-5 uM, 5-10 uM, 10-25 uM, 25-50 uM, or 50 uM or more. In various embodiments, the p53 inhibitor is Pifithrin-alpha. For example, this includes a media formulated as follows: 5 mL FBS, 500 uL ITS (100×), 50 uL Fungizone, 500 uL Pen/Strep (100×), DMEM/F12 up to 50 mL, 5 uL SB431542, Pifithrin-alpha 10 uM, ROCKi (Y-27632 dihydrochloride)—10 uM. Also described herein is a quantity of organoids including cells made by the aforementioned methods. In various embodiments, the organoids include proximal and/or distal cells. In various embodiments, the distal cells are alveolar type II progenitors and/or Scgb1a1 positive club progenitor cells.

Also described herein is a method of treatment, including administering to a human subject afflicted with disease and/or condition, a composition including a quantity of human stem cells or stem cell derived cells, wherein the quantity of human stem cells or stem cell derived cells have been cultured in the presence of at least one molecule including: a RHO-kinase (ROCK) inhibitor, SMAD inhibitor, and p53 inhibitor prior to administering to the human subject. In various embodiments, the disease and/or condition affects lung, liver, kidney and intestine, further including epithelium of the aforementioned organs, tissue and cells thereof. In various embodiments, the method includes administering to a human subject afflicted with cystic fibrosis, a composition including a quantity of human stem cells or stem cell derived cells, wherein the quantity of human stem cells or stem cell derived cells have been cultured in the presence of at least one molecule including: a RHO-kinase (ROCK) inhibitor, SMAD inhibitor, and p53 inhibitor prior to administering to the human subject. In other embodiments, the RHO-kinase inhibitor is Y27632 and the p53 inhibitor is Pifithrin α. In other embodiments, the stem cells are adult stem cells. In other embodiments, the stem cell derived cells are lung epithelial cells. In other embodiments, administering to a human subject includes orthotopic transplantation.

Described herein is a cell culture media. In various embodiments, the media includes at least one molecule including: a RHO-kinase (ROCK) inhibitor, SMAD inhibitor, and p53 inhibitor. In various embodiments, RHO-kinase (ROCK) inhibitor is Y-27632. IN various embodiments, the SMAD inhibitor is SB431542. In various embodiments, the aforementioned inhibitors are at a concentration of 0.1-1 uM, 1-5 uM, 5-10 uM, 10-25 uM, 25-50 uM, or 50 uM or more. In various embodiments, the p53 inhibitor is Pifithrin-alpha. For example, this includes a media formulated as follows: 5 mL FBS, 500 uL ITS (100×), 50 uL Fungizone, 500 uL Pen/Strep (100×), DMEM/F12 up to 50 mL, 5 uL SB431542, Pifithrin-alpha 10 uM, ROCKi (Y-27632 dihydrochloride)—10 uM.

### EXAMPLES

### Example 1

### Exposure to Low Energy Ionizing Radiation (X-Rays) Leads to Loss of Epithelial Progenitor Cell Function

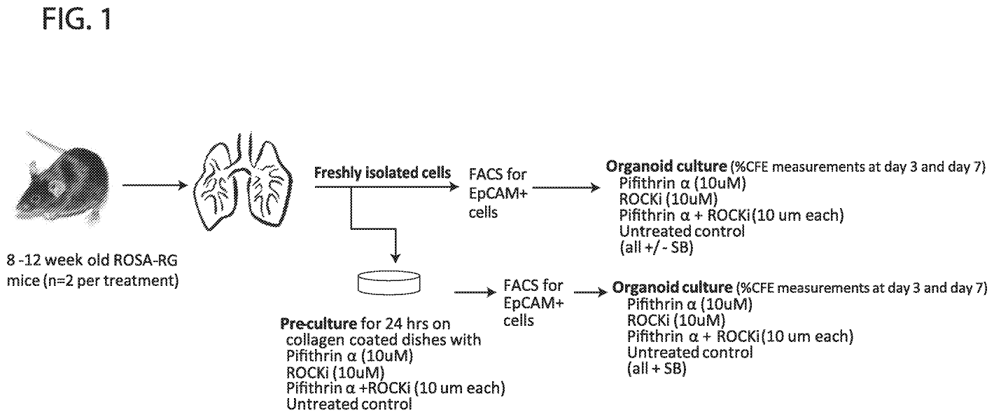

In previous work the Inventors have investigated the impact of low and high linear energy transfer (LET) ionizing radiation on the function and behavior of epithelial progenitor cells. A key finding from this work that will be exploited in the current proposal is the use of ionizing radiation to pre-condition recipient lung tissue to favor engraftment and expansion of transplanted epithelial stem cells over endogenous resident stem cells. Mice were exposed to total body irradiation (TBI) with 320 kVp X-Rays and progenitor cell function evaluated 24 hours later by FACS isolation of epithelial cells and analysis of colony-forming efficiency in 3D organoid assays (FIG. 1). Assays were performed by mixing equal numbers of epithelial cells from un-irradiated (ROSA-GFP, green) and irradiated (ROSA-RFP, red) mice, mixing with stromal support cells and evaluation of colony-forming ability following polymerization in a 3D MatriGel matrix. Relatively low doses of X-Rays resulted in loss of epithelial colony-forming ability with doses of 6 Gy and above leading to >95% reduction. The Inventors have shown that this loss of progenitor cell function does not lead to epithelial cell death (necrosis or apoptosis) and is transient with use of low LET ionizing radiation, with full restoration of progenitor cell function after 30 days. Based upon these data a dose of 6 Gy X-Ray exposure was selected for stereotactic (thoracic) pre-conditioning of recipient mice.

### Example 2

### Orthotopic Cell Transplantation

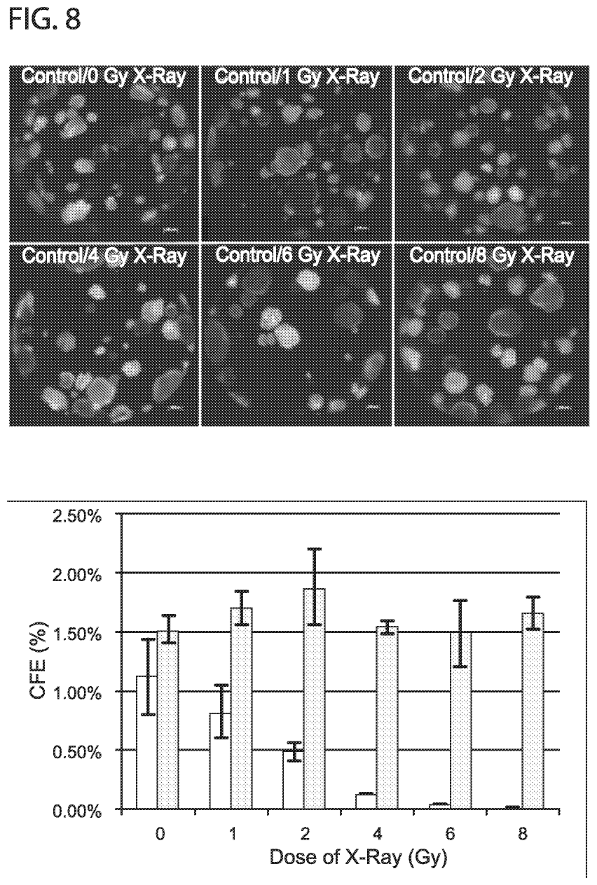

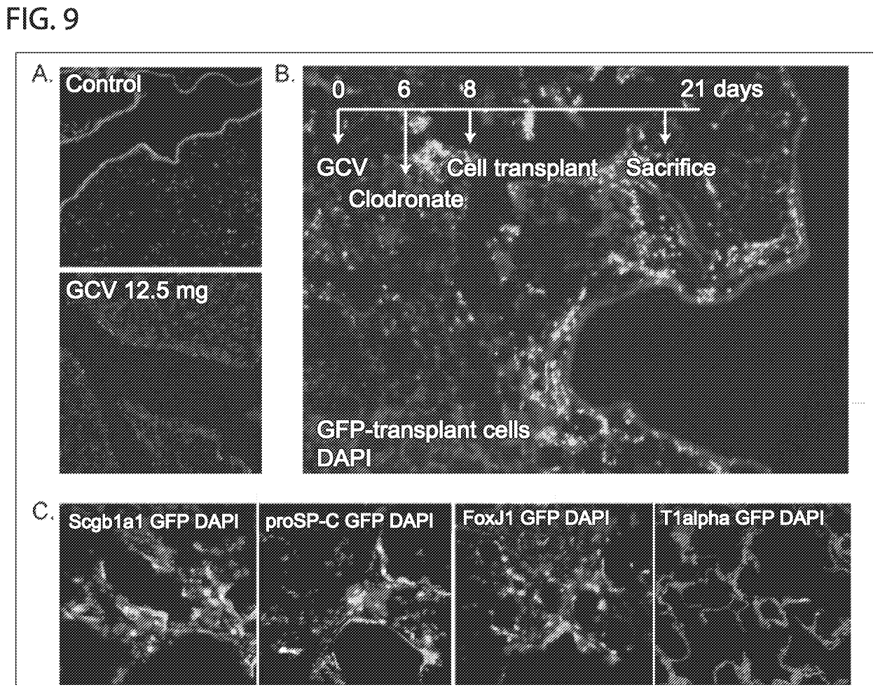

The Inventors' initial efforts to develop a protocol for functional engraftment of transplanted epithelial stem cells involved sequential ablation of club cells through delivery of gancyclovir to Scgb1a1-HSVtk trangenic mice followed by delivery of liposome encapsulated chlodronate to deplete macrophages (FIG. 9A-C). The Inventors found that only mixed populations of ROSA-GFP labeled dissociated lung tissue gave rise to engrafting epithelial cells that showed evidence of differentiation into specialized regional cell types (FIG. 9C). Difficulties with this model were lack of translatability and poor survival of recipient mice. To overcome these difficulties, the Inventors adopted low LET ionizing radiation pre-conditioning to promote engraftment of transplanted lung cells. Exposure to 6 Gy X-Rays depleted >95% of functional epithelial progenitor cells within recipient tissue (FIG. 8) and was sufficient to allow efficient engraftment of transplanted cells (FIG. 9D). Engrafting cells yielded expanding patches that were visualized by whole-mount light sheet microscopy 2 weeks post-transplant.

### Example 3

### Loss of p53 Function Enhances Colony-Forming Efficiency of Cultured Lung Progenitor Cells

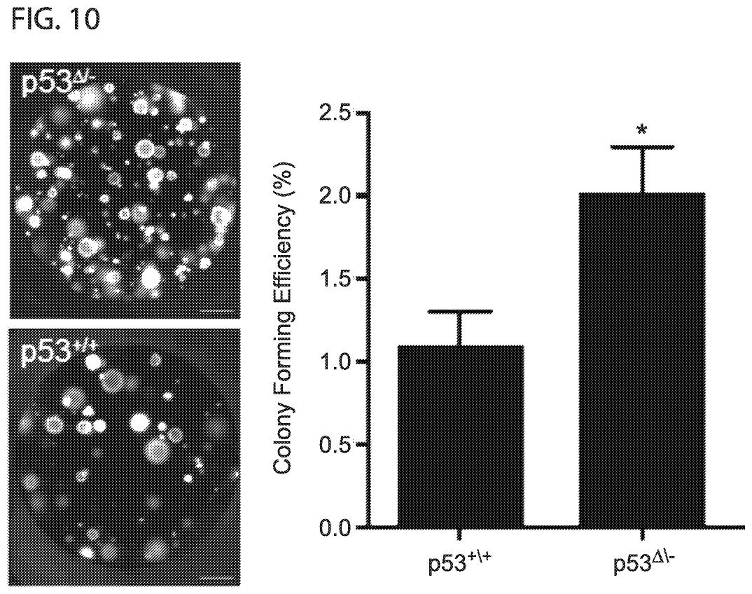

Mice were generated allowing tamoxifen-dependent lineage labeling of Scgb1a1+ club cells (Scgb1a1-CreER/ROSAmTmG) alone or together with conditional p53 loss-of-function (p53^{flox/flox}). Isolated lineage-labeled cells were placed in culture and evaluated for colony-forming efficiency and clonogenic capacity. Results shown in FIG. 10 demonstrate that p53 LOF significantly enhances club cell colony-forming efficiency expansion. The Inventors will use lineage tracing in mouse models to fate map different populations of lung epithelial cells and combine with single cell RNA-Seq to reveal their fate following transplantation compared to their native state. Epithelial stem/progenitor cells to be evaluated will include those that can be lineage labeled using either Sox2-CreER, Scgb1a1-CreER, Krt5-CreER and Sftpc-CreER, using FoxJ1-CreER as a negative control. These drivers target overlapping populations of epithelial cells and lineage trace all known epithelial stem and progenitor cells including lineage-negative/Sox2+ epithelial progenitors that are activated in response to viral infection. Lineage labeled cells will be mixed with unlabeled total lung structural cells (depleted of CD45+ cells) prior to transplantation due to the need for poorly characterized co-engrafting cells for efficient epithelial engraftment. Using this approach the Inventors will be able to evaluate the engraftment efficiency of the same number of basal, club and AT2 cells to facilitate comparison between groups using each of the endpoints indicated below. Lungs of recipient mice will be injured (pre-conditioned) to facilitate donor cell engraftment by stereotactic exposure of the thorax to ionizing radiation (X-Rays).

The Inventors have shown that either low or high energy ionizing radiation acutely depletes epithelial progenitor cell pools in lungs of mice following total body exposure and that radiation pre-conditioning facilitates engraftment and expansion of transplanted lung cells. The Inventors propose to use state-of-the-art lineage tracing, single cell RNA-Seq and orthotopic transplantation models to address unanswered questions regarding the identity and fate of engrafting cells that must be addressed to fully understand the therapeutic potential for cell-based transplantation. The Inventors expect that knowledge gained in these studies will benefit other consortium members interested in transplantation of either postnatal or iPSC-derived lung stem cells.

### Example 4

### Identity and Fate of Engrafting Epithelial Stem Cells

Cre “driver” mouse lines expressing CreER^{T2} under the regulatory control of endogenous Sox2, Scgb1a1, Krt5, Sftpc and Foxj1 genes will be established in a background that is compound heterozygous at the ROSA26 locus for the ROSA-R-tdT Cre reporter and ROSA-Luc/GFP alleles (ROSA^{R-tdT/R-Luc}) to allow tamoxifen-dependent lineage tracing of conducting airway epithelium, club, basal, alveolar type 2 and ciliated epithelial cells, respectively, with both tdTomato and luciferase. Experimental mice heterozygous for one of the five Cre driver loci and compound heterozygous for ROSA^{R-tdT/R-Luc} will be treated with 3×200 αg/g (one dose delivered every other day for a total of 3 doses) dissolved in corn oil (20 mg/ml and sonicated at 37° C. until fully solubilized) for introduction of lineage tags. Similarly exposed groups will be used either for isolation of donor lung cells to follow the fate of lineage traced cells after transplantation, or to follow the fate of lineage traced cells in the absence of lung tissue perturbation/transplantation. Donor lung cells will be prepared 2 days after the final tamoxifen dose using a standardized dissociation protocol involving mechanical and enzymatic treatment to generate single cell suspensions of total lung or tracheal cells depending upon the lineage trace used The lineage-labeled (RFP+) epithelial cell fraction (CD31−, CD45−, CD326+) will be isolated by FACS and 1×10⁵ fractionated cells mixed with 1×10⁶ total luffng cells that have been depleted of CD45+ cells by magnetic bead separation. Donor cells will be delivered to lungs of pre-conditioned adult male C57Bl/6J mice by intratracheal instillation in 50 μl sterile saline. Radiation pre-conditioning will be achieved by delivery of 6 Gy X-Rays to the thorax of immobilized mice using an XRadSmart stereotactic irradiator equipped with microCT for mapping and guidance (Precision X-Ray). Endpoints evaluated among transplant recipients and controls are summarized in Table 1.

TABLE 1
Endpoints to be evaluated for
assessment of stem cell engraftment.

| Endpoint | Bioluminescent | | Light sheet | | | Single cell |
|---|---|---|---|---|---|---|
| Time pt. | imaging | | microscopy | Histopath. | FACS | RNA-Seq |
| 1 week | Yes | Yes | Yes | | Yes | |
| 2 weeks | Yes | Yes | | | | |
| 4 weeks | Yes | Yes | Yes | Yes | Yes | |
| 8 weeks | Yes | Yes | | | | |
| 12 weeks | Yes | Yes | Yes | Yes | Yes | Yes |

Transplant recipients will be monitored weekly for engraftment and expansion of luciferase-positive cells by delivery of D-luciferin (150 mg/kg in saline, i.p.) and bioluminescent imaging using an XRadSmart. Lung tissue will be harvested from transplant recipients and lineage traced control mice. One cohort from each group (n=5, Grey in Table 1) will be fixed, treated with scale to clarify tissue and processed for sequential whole-mount light sheet microscopy followed by paraffin embedding and immunofluorescence detection of engrafting cells (GFP+/RFP−=donor-derived non-lineage-labeled, GFP+/RFP+=donor-derived lineage-labeled). A second cohort from each group (n=5, Blue in Table 1) will be processed for isolation of total lung single cells, stained for surface CD45, CD31, and CD326, and evaluated by flow cytometry to 1) identify and quantify engrafting cells, and 2) for sorting of lineage-positive (both control and transplant recipients) and -negative (transplant recipients only) donor-derived cells for evaluation by 10× Chromium single cell RNA-Seq (each cell fraction will be independently bar-coded and pooled according to groups for sequencing by Nova-Seq).

### Example 5

### Clonal Behavior of Engrafting Epithelial Stem Cells

Cre driver lines will be identical, with the exception that the ROSA locus will be ROSA^{R-Confetti/R-Luc} in place of ROSAR-tdT/R-Luc. Tamoxifen exposures, cell isolation and transplantation into pre-conditioned recipients. Endpoints will be those shown in grey in Table 1, without the use of the non-transplanted control group. Light sheet microscopy will be used to image nGFP, cYFP, cRFP and mCFP to assess clonality of engrafting cells. Histopathology coupled with immunofluorescence detection of GFP variants (nGFP, cYFP and mCFP) and RFP will be used to verify clonality and fate of engrafting cells by coupling with cell type-specific markers including Scgb1a1 (club), Krt5 (basal), Foxj1 (ciliated), Sftpc (AT2) and Pdpn (AT1 and basal).

It is expected that lineage committed epithelial progenitor cells will engraft following transplantation, generating specialized progeny consistent with their identity within donor tissue. The Inventors will determine whether airway progenitor cell types (Krt5+, Scgb1a1+, Sox2+) have a propensity for engraftment and/or expansion within airway rather than alveolar microenvironments and whether alveolar progenitor cells (Sftpc+) preferentially engraft and/or expand within alveolar rather than airway microenvironments. It is unlikely that measurable engraftment will occur within mismatched, sub-optimal, anatomic locations (airway epithelial stem cells in alveoli and vice versa). Through mapping these events for each population of lineage-labeled epithelial cells the Inventors will be able to determine their fate and clonal ability following engraftment within either matched or mismatched microenvironments. Light sheet microscopy will provide the most accurate data for determination of the spatial context of engrafting cells and clonal expansion, with immunofluorescence of histological sections providing data on immunophenotype of engrafting cells.

It is not likely that that epithelial stem cells will contribute to formation of cell types of non-epithelial lineages (i.e. they will lack multipotency). This will be verified by flow cytometry using lineage-specific cell surface markers in combination with reporters for total engrafting cells and lineage-traced cells, in addition to single cell RNA-Seq of the lineage-labeled epithelial population. Comprehensive single cell transcriptome profiling will allow us to determine the fate of each lineage-labeled epithelial cell type in their native tissue environment (control, uninjured mice) compared to the microenvironment post-transplantation. The Inventors expect that transplanted cells, if they have the capacity to engraft, will do so in both matched and mismatched tissue microenvironments, and that the fate of engrafting cells will be determined through a combination of intrinsic and microenvironmental factors. Molecular similarity between control and transplanted lineage-labeled populations will be evaluated by t-distributed stochastic neighbor embedding (tSNE) analysis and used as a measure of epithelial cell fate; equivalent cell fates, such as between control lineage-labeled cells versus transplanted cells engrafting within matched microenvironments, will overlap by tSNE and share similar molecular phenotypes as revealed in heat maps of transcriptomes. In contrast, transplanted cells that assume different fates from their control lineage-labeled counterparts will map distantly by tSNE and show marked differences in relative gene expression revealed in heat maps. Pathway analysis of differentially expressed genes will be used to infer altered signaling leading to divergent cell fates. Future studies that are beyond the scope of this application will investigate the fate of multipotent lung endodermal progenitor cells recovered from pooled E12.5 mouse lung to determine whether their fate is determined by microenvironment (site of engraftment) without the influence of pre-determined intrinsic cell fates seen with adult region-specific stem cells.

Inefficient engraftment and/or expansion of some or all the lineage traced cell types can result in difficulties in recovery of sufficient cell numbers for single cell RNA-Seq using the 10× chromium platform (a minimum of 1×10⁴ cells are needed). If this is the case, lineage-labeled cells will be processed using the Smart-Seq protocol in which single cells are sorted into wells of 384 well plates and barcoded single cell libraries prepared and pooled. This protocol would yield considerably fewer cells for analysis but would provide data for rare engraftment events.

### Example 6

### Lineage Tracing to Define Cellular Interactions that Promote Epithelial Stem Cell Engraftment

Engraftment of highly enriched lung epithelial progenitor cells is significantly attenuated compared to mixed populations of unfractionated lung cells. These findings are also consistent with observations in culture models, for which co-cultured lung stromal cells are required for expansion of isolated epithelial stem cells to yield clonally-derived organoids. These data suggest that engraftment is dependent either upon direct cell-cell interactions, most likely the result of aggregation, and/or that paracrine factors from co-engrafting cells promote survival, engraftment or clonal expansion of epithelial stem cells.

Experiments use lineage tracing to systematically investigate roles played by lung stromal and endothelial cells in promoting engraftment and clonal expansion of epithelial stem/progenitor cells. Structural lung cells will be lineage labeled using Shh-Cre, Pdgfb-Cre or Tbx4-Cre to efficiently trace epithelial, endothelial and stromal cell types, respectively. Dissociated lung cells will be transplanted as either mixed populations without fractionation, individual fractionated populations, or reconstituted fractionated cell populations. The Inventors will determine the impact of cell sorting on engraftment potential and viability, and the ability to reconstitute critical cellular interactions through recombination of fractionated cells. These data will not only provide critical insights into cellular interactions that promote engraftment of transplanted lung cells, but determine the feasibility of recombining stem cells from other sources, such as iPSC-derived lung endoderm or in vitro expanded epithelial stem cells, with co-engrafting structural lung cells to boost engraftment efficiency.

### Example 7

### Identity and Fate of Co-Engrafting Structural Lung Cells

Cre “driver” mouse lines expressing constitutively active Cre under the regulatory control of either Shh, Tbx4 or Pdgfb promoter elements will be established in a background that is compound heterozygous at the ROSA26 locus for the ROSA-R-tdT Cre reporter and ROSA-Luc/GFP alleles (ROSA^{R-} tdT/LucGFP). These lines will allow lineage tracing of epithelial, stromal and endothelial cell types, respectively, in a ubiquitous luciferase/GFP background. Mice that are heterozygous for one of the three Cre drivers and compound heterozygous for ROSA^{R-tdT/LucGFP} will be used for cell isolation, fractionation and orthotopic transplantation. Donor lung cells will be prepared by dissociation of lung and tracheal tissue to generate single cell suspensions. CD45-magnetic beads will be used for depletion of CD45+ cells and remaining “structural” cells will transplanted into either PR8 or X-Ray pre-conditioned hosts either as 1) unsorted mixed populations, 2) following FACS depletion of each of the lineage-labeled cell populations, 3) FACS enriched Shh (epithelial) lineage cells, or 3) following reconstitution of all three lineages as a “recombined” mixed population. Donor cells will be delivered to lungs of preconditioned adult male C57Bl/6J mice (n=5 per cohort for a total of 10 mice per group) by intratracheal instillation of 1×10⁶ cells in 50∝1 sterile saline. Engraftment and expansion of transplanted cells will be monitored as shown in Table 2, using bioluminescent imaging, histopathology coupled with immunofluorescence detection and immunophenotyping of engrafting cells, and by FACS analysis of dissociated recipient lungs to follow their fate post transplantation.

TABLE 2
Endpoints to be evaluated for assessment of stem cell engraftment

| Endpoint | Bioluminescent | | | |
|---|---|---|---|---|
| Time pt. | imaging | | Histopath. | FACS |
| 1 week | Yes | Yes | Yes | Yes |
| 4 weeks | Yes | Yes | | |
| 8 weeks | Yes | Yes | | |
| 12 weeks | Yes | Yes | Yes | Yes |

All mouse lines needed to complete this aim are available as in-house breeding colonies and the Inventors do not anticipate technical difficulties in completion of experiments. Proposed studies represent a significant improvement over preliminary and published work in that lineage tracing will be coupled with FACS enrichment/depletion strategies for selection of desired cell populations. The Inventors will determine what structural cells are required to promote engraftment and expansion of engrafting epithelial stem cells following orthotopic transplantation. The Inventors expect efficient engraftment of epithelial stem cells from total mixed structural lung cells and that highly enriches Shh lineage cells will lack engraftment potential. The requirement for both stromal and endothelial co-engrafting cells will be determined as will the fate of these co-engrafting cells (i.e. whether they reconstitute non-epithelial cell types of the regenerating lung). Finally, the Inventors will determine whether fractionated single cells that correspond to epithelial (Shh lineage), stromal (Tbx4 lineage) and endothelial (Pdgfb lineage) can be functionally recombined to restore engraftment potential of epithelial stem cells.

### Example 8

### Pharmacologic Manipulation to Enhance Functional Integration of Transplanted Epithelial Progenitors

Transient pharmacological modulation of pathways that promote survival and renewal capacity of transplanted epithelial cells can overcome the requirement for co-transplanted (non-epithelial) cells for their efficient engraftment and expansion. Overcoming the requirement for delivery of co-transplanted cells will reduce the risk of structural lung remodeling that could otherwise result from inclusion of stromal cell types and pave the way for efficient transplantation of autologous adult or iPSC-derived lung epithelial cell types. Lineage-labeled lung and tracheal epithelium will be pre-treated by drugs that block signaling by Rho kinase or p53 24 hours prior and during isolation from donor tissue. Even though it is well recognized that chronic modulation of Rho kinase and p53 signaling is associated with neoplasia, the Inventors will use transient modulation of these critical fate-determining signaling molecules to overcome the adverse effects of cellular stress that accompany tissue dissociation and transplantation, on long-term functional engraftment. Pharmacological inhibition of ROCK1 and 2 by the small molecule Y27632 has been shown to promote survival and expansion of human bronchial epithelial stem cells, and represent a good candidate for pre-treatment of freshly isolated lung epithelial stem cells prior to transplantation. Similarly, the Inventors' previous work has shown that conditional genetic loss of p53 function enhances both “sternness” and colony-forming ability of mouse airway club cells, and that pharmacologic inhibition of p53 by the small molecule pifithrin-αconfers protection against p53-induced neuronal cell death. The Inventors will pharmacologically target these key regulators of cell survival and fate with the goal of short-term modulation of epithelial survival and “sternness” without the potential for long-term adverse effects such as neoplasia. Epithelial progenitor cells treated transiently with ROCK and p53 inhibitors will be characterized in vitro using 3D organotypic assays to determine effects on colony-forming ability and following orthotopic transplantation into preconditioned syngeneic recipient mice to assess engraftment potential and fate.

### Example 9

### Impact of Pharmacological Inhibitors of Rho Kinase and p53 on In Vitro 3D Epithelial Colony-Forming Ability

Lineage-labeled lung epithelium will be prepared from donor mice (n=5 per group) that are heterozygous for Shh-Cre and compound heterozygous at the ROSA26 locus for the ROSA-R-tdT Cre reporter and ROSA-Luc/GFP alleles (ROSA^{R-}tdT/LucGFP). Donor mice will receive i.p. injections of either saline (control), Y27632 (10 mg/kg) or pifithrin-a (2 mg/kg) 24 hours prior to harvesting lung+tracheal tissue. Media for tissue dissociation and cell fractionation will be supplemented with the corresponding small molecules for drug treated groups, either Y27632 (10 μM) or pifithrin-α (2 using media alone for the control cells, and viable lineage-labeled CD326+ lung epithelial cells isolated by FACS using a BD Influx sorter. Yield of epithelial cells will be 6×10⁵-8×10⁵, of which triplicate cultures will be prepared, using previously optimized methods, by recombining either 500 or 2,000 epithelial cells with 1×10⁵ MLg fibroblasts and polymerizing in 50% growth factor depleted MatriGel in 24 well Transwells. Remaining cells from each group will be used for transplantation experiments. Cultures will be maintained for 2 weeks to determine colony-forming efficiency of lineage-labeled epithelial cells and for quantification of bioluminescence, as measures of clonogenic capacity.

### Example 10

### Impact of Pharmacological Inhibitors of Rho Kinase and p53 on Engraftment Potential, Clonogenic Capacity and Fate of Transplanted Epithelial Stem Cells

Epithelial progenitor cells are transplanted into PR8 and X-Ray pre-conditioned hosts. Pre-conditioning will be performed as detailed above and 2×10⁵ enriched epithelial cells from each donor mouse delivered to paired PR8 and X-Ray treated recipients. Controls will be prepared from untreated cells and processed to yield either mixed populations of lung and tracheal cells that have been depleted of CD45+ hematopoietic cells only (mixed structural cells) or fractionated to yield lineage-labeled CD326+ cells as for inhibitor treated samples. Either 1×10⁶ or 2×10⁵ control cells will be delivered for mixed structural cells or fractionated lineage-labeled epithelial cells, respectively. Engraftment and expansion of transplanted lineage-labeled cells will be monitored longitudinally by bioluminescent imaging and by flow cytometry and single cell RNA-Seq 12 weeks post-transplant to assess fate of transplanted lineage-labeled epithelial cells (Table 3).

TABLE 3
Endpoints to be evaluated for assessment
of stem cell engraftment following
transient pharmacological inhibition
of ROCK or p53 in enriched lung epithelial cells

| Endpoint | Bioluminescent | | Single cell |
|---|---|---|---|
| Time pt. | imaging | FACS | RNA-Seq |
| 1 week | Yes | | |
| 4 weeks | Yes | | |
| 8 weeks | Yes | | |
| 12 weeks | Yes | Yes | Yes |

The Inventors expect that anoikis and loss of clonogenic potential are major impediments to efficient engraftment and clonal expansion of transplanted epithelial stem cells. Accordingly, the Inventors expect that transient inhibition of ROCK and p53 pathways will dramatically enhance engraftment and promote initial clonal expansion of transplanted epithelial stem cells. Using bioluminescent imaging of transplant recipients the Inventors will monitor engraftment and expansion as a function of both pharmacologic pre-treatment of donor cells and pre-conditioning regiment applied to recipient mice. The Inventors expect that transient inhibition of ROCK and p53 will each lead to enhanced bioluminescence compared to transplants of untreated epithelial cells. Comparison will be made to transplant of unfractionated lung structural cells, ROCK inhibition and p53 inhibition, both at initial transplantation and as a function of time post-transplant.

It is suggested that the principal benefit of ROCK/p53 inhibition will be at the time of engraftment and that chronic inhibition of ROCK/p53 among recipients could lead to adverse outcomes including tissue remodeling and neoplasia. The Inventors have used in vitro organoid assays to validate the impact of pharmacologic inhibition of p53 on the clonogenic potential of lung stem cells. The Inventors have found that effects of the p53 inhibitor pifithrin-α are additive when used in combination with other drugs that modulate stem cell expansion (ROCK inhibitors and TGFb inhibitors). The Inventors' ongoing work seeks to verify that increased clonogenic potential imparted by this drug is truly associated with stem cell expansion, that expanded lung stem cells retain their full capacity to generate specialized lung cell types and that p53 is the direct target of pifithrin-α that mediated its effects on stem cells.

### Example 11

### Additional Results

ROCKi increases the colony forming efficiency when murine distal total epithelial cells (isolated from the lung without trachea) are pre-cultured on collagen coated plates for 24 hrs prior after initial isolation. The effect on size is more pronounced when cells are precultured for 24 hrs on collagen coated plates after initial isolation. The drop in CFE in the Pifithrin alpha+ROCKi group is likely due space and nutrient availability constraints due to the large number of organoids per well. Addition of both ROCKi and Pifithrin alpha (in presence of SB431542) increases the colony forming efficiency of freshly isolated alveolar type II cells (regional stem cells which maintain the epithelium of the air sacs in the lung). The effect is synergistic for their combined use, similar to that seen for total distal epithelial cells in the previous figures. Addition of both ROCKi and Pifithrin alpha does not increase the size of the alveolar organoids

Distal progenitor cells were cultured as 3D organoids for 14 days in presence of Pifithrin-alpha, ROCKi and SB431542 followed by culture for 7 days in media without all three inhibitors. Addition of inhibitors did not alter the differentiation potential of the cells. Addition of ROCKi (in presence of SB431542) increases the colony forming efficiency of freshly isolated club cells (regional stem cells which maintain certain cell types in the bronchiolar epithelium) whereas addition of Pifithrin alpha (in presence of SB431542) increases the size of Scgb1a1-derived organoids. Addition of both ROCKi (in presence of SB431542) increases the colony forming efficiency of freshly isolated murine proximal total epithelial cells (isolated from the murine trachea). However, addition of pifithrin alpha does not show a significant effect. Thus ROCKi seems to increase survival of both proximal and distal progenitors while the effect of Pifithrin alpha is specific to distal stem cells.

In view of the aforementioned, addition of Pifithrin α causes an increase in organoids size, while addition of ROCKi causes an increase in the colony forming efficiency. This effect is more pronounced when cells are pre-cultured for 24 hrs on collagen coated plates after initial isolation. Current results indicate that overall, ROCKi increases survival of progenitor cells as indicated by the increase in colony forming ability of all proximal and distal progenitor cell types. This effect of p53 inhibition is more pronounced on distal progenitor cells in comparison to proximal progenitor cells.

Within the distal progenitor sub-types, p53 inhibition increases the survival of alveolar type II progenitors (indicated by increased % CFE) and proliferation of Scgb1a1 positive club progenitor cells (indicated by increased organoid size).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to expansion of stem cells, including adult stem cells such as epithelial airway cells, small molecules, methods and compositions related to use of the aforementioned compositions, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of culturing cells, comprising:
providing a quantity of human stem cells, or stem cell derived cells;
culturing in the presence of at least one molecule comprising: a RHO-kinase (ROCK) inhibitor, SMAD inhibitor, and p53 inhibitor; and
expanding the quantity of stem cells, or stem cell derived cells.

2. The method of claim 1, wherein the stem cells or stem cell derived cells are added to a media comprising at least one molecule comprising: a RHO-kinase (ROCK) inhibitor, SMAD inhibitor, and p53 inhibitor.

3. The method of claim 1, wherein the RHO-kinase inhibitor is Y27632.

4. The method of claim 1, wherein the p53 inhibitor is Pifithrin α.

5. The method of claim 1, wherein the SMAD inhibitor is SB 431542.

6. The method of claim 1, wherein the stem cells are adult stem cells.

7. The method of claim 1, wherein the stem cell derived cells are lung epithelial cells.

8. The method of claim 1, wherein the stem cell derived cells are organoids.

9. The method of claim 1, wherein expanding the quantity of stem cells or stem cell derived cells comprises increased proliferation of the cells.

10. The method of claim 1, wherein expanding the quantity of stem cells or stem cell derived cells comprises increased size of organoids comprising stem cell derived cells.

11. The method of claim 1, wherein the expanding the quantity of stem cells or stem cell derived cells comprises increased colony forming efficiency.

12. The method of claim 1, wherein the at least one molecule comprises Y27632 and Pifithrin α.

13. A cell culture comprising:
a quantity of human stem cells, or stem cell derived cells in a cell culture media comprising at least one molecule comprising: a RHO-kinase (ROCK) inhibitor, SMAD inhibitor, and p53 inhibitor.

14. The cell culture of claim 13, wherein the RHO-kinase inhibitor is Y27632 and the p53 inhibitor is Pifithrin α.

15. The method of claim 13, wherein the stem cells are adult stem cells.

16. The method of claim 13, wherein the stem cell derived cells are lung epithelial cells.

17. A method of treatment, comprising:
administering to a human subject afflicted with cystic fibrosis, a composition comprising a quantity of human stem cells or stem cell derived cells, wherein the quantity of human stem cells or stem cell derived cells have been cultured in the presence of at least one molecule comprising: a RHO-kinase (ROCK) inhibitor, SMAD inhibitor, and p53 inhibitor prior to administering to the human subject.

18. The method of claim 17, wherein the RHO-kinase inhibitor is Y27632 and the p53 inhibitor is Pifithrin α.

19. The method of claim 17, wherein the stem cells are adult stem cells.

20. The method of claim 17, wherein the stem cell derived cells are lung epithelial cells.
